SEQUENCE LISTING (4 sequences)

SEQ ID NO: 1 - protein, 1117 aa, Artificial Sequence
  Description of Artificial Sequence Note = Synthetic Construct
MADGPRCKRRKQANPRRNNVTNYNTVVEANSDSDDEDKLHIVEEESITDAADCEGGMPDDELPADQTVLPGGSDRGGGAKNCWQDNVKDNECDSDAENEQNHDPNVEEFLQQQDTAVIYPEAPEEDQRQGTPEASSHDENGTPDAFSQLLTCPYCDRGYKRFTSLKEHIKYRHEKNEDNFSCSLCSYTFAYRTQLERHMTSHKSGREQRHVTQSGGNRKFKCTECGKAFKYKHHLKEHLRIHSGEKPYECPNCKKRFSHSGSYSSHISSKKCISLMPVNGRPRSGLKTSQCSSPSLSTSPGSPTRPQIRQKIENKPLQEPLSVNQIKTEPVDYEFKPIVVASGINCSTPLQNGVFSSGGQLQATSSPQGVVQAVVLPTVGLVSPISINLSDIQNVLKVAVDGNVIRQVLETNQASLASKEQEAVSASPIQQGGHSVISAISLPLVDQDGTTKIIINYSLEQPSQLQVVPQNLKKEIPAPTNSCKSEKLPEDLTVKSETDKSFEGARDDSTCLLCEDCPGDLNALPELKHYDPECPAQPPPPAPATEKPESSASSAGNGDLSPSQPPLKNLLSLLKAYYALNAQPSTEELSKIADSVNLPLDGVKKWFEKMQAGQIPGQSPDPPSPGTGSVNIPTKTDEQPQPADGNEPQEDSTRGQSPVKIRSSPVLPVGSAMNGSRSCTSSPSPLNLCSARNPQGYSCVAEGAQEEPQVEPLDLSLPKQQGELLERSTVSSVYQNSVYSVQEEPLNLSCAKKEPQKDSCVTDSEPVVNVVPPSANPINIAIPTVTAQLPTIVAIADQNSVPCLRALAANKQTILIPQVAYTYSATVSPAVQEPPVKVIQPNGNQDERQDTSSEGVSTVEDQNDSDSTPPKKKTRKTENGMYACDLCDKIFQKSSSLLRHKYEHTGKRPHECGICRKAFKHKHHLIEHMRLHSGEKPYQCDKCGKRFSHSGSYSQHMNHRYSYCKRGAEDRDAMEQEDAGPEVLPEVLATEHVGARASPSQADSDERESLTREEDEDSEKEEEEEDKEMEELQEGKECENPQGEEEEEEEEEEEEEEEEEEEVEADEAEHEAAAKTDGTVEVGAAQQAGSLEQKASESEMESESESEQLSEEKTNEA

SEQ ID NO: 2 - protein, 708 aa, Artificial Sequence
  Description of Artificial Sequence Note = Synthetic Construct
MADGPRCKRRKQANPRRNNVTNYNTVVEANSDSDDEDKLHIVEEESITDAADCEGGMPDDELPADQTVLPGGSDRGGGAKNCWQDNVKDNECDSDAENEQNHDPNVEEFLQQQDTAVIYPEAPEEDQRQGTPEASSHDENGTPDAFSQLLTCPYCDRGYKRFTSLKEHIKYRHEKNEDNFSCSLCSYTFAYRTQLERHMTSHKSGREQRHVTQSGGNRKFKCTECGKAFKYKHHLKEHLRIHSGEKPYECPNCKKRFSHSGSYSSHISSKKCISLMPVNGRPRSGLKTSQCSSPSLSTSPGSPTRPQIRQKIENKPLQEPLSVNQIKTEPVDYEFKPIVVASGINCSTPLQNGVFSSGGQLQATSSPQGVVQAVVLPTVGLVSPISINLSDIQNVLKVAVDGNVIRQVLETNQASLASKEQEAVSASPIQQGGHSVISAISLPLVDQDGTTKIIINYSLEQPSQLQVVPQNLKKEIPAPTNSCKSEKLPEDLTVKSETDKSFEGARDDSTCLLCEDCPGDLNALPELKHYDPECPAQPPPPAPATEKPESSASSAGNGDLSPSQPPLKNLLSLLKAYYALNAQPSTEELSKIADSVNLPLDGVKKWFEKMQAGQIPGQSPDPPSPGTGSVNIPTKTDEQPQPADGNEPQEDSTRGQSPVKIRSSPVLPVGSAMNGSRSCTSSPSPLNLCSARNPQGYSCVAEGAQEEP

SEQ ID NO: 3 - protein, 882 aa, Artificial Sequence
  Description of Artificial Sequence Note = Synthetic Construct
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 4 - protein, 708 aa, Artificial Sequence
  Description of Artificial Sequence Note = Synthetic Construct
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIP

(19) United States
(12) Patent Application Publication
(10) Pub. No.: US 2011/0165698 A1
(43) Pub. Date: Jul. 7, 2011

(54) COMPOSITIONS AND METHODS RELATING TO DETECTION OF SOLUBLE E-CADHERIN IN NEURODEGENERATIVE DISEASE

(76) Inventors: Sabine M. Brouxhon, Stony Brook, NY (US); Stephanos Kyrkanidies, Stony Brook, NY (US); Ross H. Tallents, Rochester, NY (US); M. Kerry O'Banion, Pittsford, NY (US)

(21) Appl. No.: 12/994,324

(22) PCT Filed: May 26, 2009
(86) PCT No.: PCT/US09/45149

§ 371 Date: Mar. 8, 2011

(30) Foreign Application Priority Data
May 23, 2008 (US) 61/055688

(51) Int. Cl. G01N 33/53; G01N 33/68

(52) U.S. Cl. 436/501; 436/87

(57) ABSTRACT

Disclosed herein are compositions and methods for diagnosing, detecting, or monitoring neural disease, conditions, or disorders involving detection of soluble E-cadherin.

### CROSS-REFERENCE TO RELATED APPLICATIONS

This application claims benefit of U.S. Provisional Application No. 61/055,688, filed May 23, 2009, which is hereby incorporated herein by reference in its entirety.

### STATEMENT REGARDING FEDERALLY SPONSORED RESEARCH

This invention was made with government support under Grant R01 NS048839, AG030149, and AG028325 awarded by the National Institutes of Health. The government has certain rights in the invention.

### BACKGROUND

There are a number of neural diseases, conditions, and disorders for which additional means of early detection, diagnosis, and monitoring are desired. For example, neurodegenerative diseases, in particular Alzheimer's disease (AD), constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

As another example, Tay-Sachs disease (abbreviated TSD, also known as GM2 gangliosidosis, Hexosaminidase A deficiency or Sphingolipidosis) is a genetic disorder, fatal in its most common variant known as Infantile Tay-Sachs disease. TSD is inherited in an autosomal recessive pattern. The disease occurs when harmful quantities of a fatty acid derivative called a ganglioside accumulate in the nerve cells of the brain. Gangliosides are lipids, components of cellular membranes, and the ganglioside GM2, implicated in Tay-Sachs disease, is especially common in the nervous tissue of the brain.

Likewise, neuronal stress can occur due to a wide range of conditions, illnesses, injuries, and as a result of iatrogenesis. Possible causes of widespread (diffuse) neuronal stress include prolonged hypoxia (shortage of oxygen), poisoning by teratogens (including alcohol), infection, and neurological illness. Chemotherapy can cause damage to the neural stem cells and oligodendrocyte cells that produce myelin. Common causes of focal or localized neuronal stress are physical trauma (traumatic brain injury), stroke, aneurysm, surgery, or neurological illness.

There is therefore a need in the art for improved compositions and methods for diagnosing, detecting, or monitoring neural disease, conditions, or disorders, such as neurodegenerative diseases and neuronal stress.

### BRIEF SUMMARY

In accordance with the purpose of this invention, as embodied and broadly described herein, this invention relates to compositions and methods for diagnosing, detecting, or monitoring neural disease, conditions, or disorders.

For example, disclosed herein is a method of diagnosing a neurodegenerative disease or neuronal stress in a subject, comprising detecting the level of soluble E-cadherin (sE-cad) in a sample from the subject and comparing it to one or more reference standards.

Also disclosed herein is a method of monitoring the severity of a neurodegenerative disease or neuronal stress in a subject, comprising comparing levels of soluble E-cadherin (sE-cad) in a sample obtained from the subject at multiple time points, wherein a change in sE-cad levels in samples at later time points indicates a change in severity of neurodegenerative disease.

Also disclosed herein is a method of monitoring a response to a neurodegenerative disease or neuronal stress treatment in a subject, the method comprising comparing levels of soluble E-cadherin (sE-cad) in a sample obtained from the subject at multiple time points during treatment of the subject, wherein a decrease in sE-cad levels in samples at later time points indicates effective treatment.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows E-cadherin transcript levels are dramatically increased in HexB^{-/-} mice. FIG. 1A shows use of semi-quantitative RT-PCR to examine levels of E-cadherin in 4 month old brains from wild-type (WT) and HexB^{-/-} mice. E-cadherin mRNA was greatly induced in 4 month old HexB^{-/-} mice compared to age-matched littermate WT controls. At 4 months of age, HexB^{-/-} mice clinically exhibit signs of neurodegeneration including muscle weakness, rigidity and motor deterioration. FIG. 1B shows WT and HexB^{-/-} brains resected at 4 months of age and processed for E-cadherin levels by Western immunoblotting.

FIG. 2 shows co-localization of E-cadherin in neurons of HexB^{-/-} mice. The identity of brain cells expressing E-cadherin in HexB^{-/-} mice was examined by double immunofluorescence. Shown is E-cadherin staining (panel A) located in a perinuclear/cytoplasmic distribution of Neun-positive (panel B) neurons. Slides were examined on an Olympus fluorescent microscope.

FIG. 3 shows immunostaining for E-cadherin (20-100× magnification) in the brains of 4 month old age-matched WT and HexB^{-/-} mice and 8 month APP mice.

FIG. 4 shows immunostaining for E-cadherin (10-20× magnification) in the brains of human Alzheimers Disease Patient's and age-matched human controls.

FIG. 5 shows enhanced E-cadherin ectodomain shedding in the urine of Tay Sachs and Alzheimers (APP) mice that exhibit signs of neurodegeneration. Urine from 4 month old Tay sachs and 8 month old Alzheimers (APP) mice was collected and tested for E-cadherin fragments (sE-cad) using an antibody specific for the extracellular epitope of E-cadherin. An 80 kDa protein was detected in the urine of these mice, indicating proteolytical shedding of the protein.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS

Disclosed herein are compositions and methods relating to the detection of soluble E-cadherein (sE-cad) in a sample from a subject for the purpose of detecting, diagnosing, monitoring, or otherwise evaluating a disease or condition.

Disclosed herein are compositions and methods relating to the detection of soluble E-cadherein (sE-cad) in a sample from a subject for the purpose of detecting, diagnosing, monitoring, or otherwise evaluating a disease or condition.

Cadherins are a class of type-1 transmembrane proteins. They play important roles in cell adhesion, ensuring that cells within tissues are bound together. They are dependent on calcium (Ca²⁺) ions to function, hence their name.

The cadherin superfamily includes cadherins, protocadherins, desmogleins, and desmocollins, and more. In structure, they share cadherin repeats, which are the extracellular Ca²⁺-binding domains. There are multiple classes of cadherin molecule, each designated with a one-letter prefix (generally noting the type of tissue with which it is associated). Cadherins within one class will bind only to themselves. For example, an N-cadherin will bind only to another N-cadherin molecule. Because of this specificity, groups of cells that express the same type of cadherin molecule tend to cluster together during development, whereas cells expressing different types of cadherin molecules tend to separate.

Different members of the cadherin family are found in different locations. E-cadherins are found in epithelial tissue; N-cadherins are found in neurons; and P-cadherins are found in the placenta. T-cadherins have no cytoplasmic domains and must be tethered to the plasma membrane.

E-cadherin (epithelial) consists of 5 cadherin repeats (EC1˜EC5) in the extracellular domain, one transmembrane domain, and an intracellular domain that binds p120-catenin and beta-catenin. The intracellular domain contains a highly-phosphorylated region vital to beta-catenin binding and therefore to E-cadherin function. Beta-catenin can also bind to alpha-catenin. Alpha-catenin participates in regulation of actin-containing cytoskeletal filaments. In epithelial cells, E-cadherin-containing cell-to-cell junctions are often adjacent to actin-containing filaments of the cytoskeleton.

E-cadherin is first expressed in the 2-cell stage of mammalian development, and becomes phosphorylated by the 8-cell stage, where it causes compaction. In adult tissues, E-cadherin is expressed in epithelial tissues, where it is constantly regenerated with a 5-hour half-life on the cell surface.

Loss of E-cadherin function or expression has been implicated in cancer progression and metastasis. E-cadherin downregulation decreases the strength of cellular adhesion within a tissue, resulting in an increase in cellular motility. Although the regulation of cadherin adhesive activity is not completely understood, the level of cadherin gene expression correlates with the strength of adhesion, and the type of cadherin expressed affects the specificity of cell interaction. Posttranscriptional mechanisms regulating cadherin adhesion include modulation of cadherin clustering at the cell surface, changes in cadherin interaction with other proteins, and proteolysis of cadherin ectodomains.

In addition to cleaving extracellular matrix proteins, MMPs mediate shedding of transmembrane and membrane-associated proteins, including E-cadherin. For example, exogenous matrilysin and stromelysin-1 (MMP-3) can release an 80-kd E-cadherin fragment from various cancer cell lines and ectopic expression of stromelysin-1 leads to cleavage of E-cadherin in mammary epithelial cells.

Thus, the accumulation of the soluble ectodomain of E-cadherin in human serum has also been associated with the progression of prostate and breast cancer and is thought to be mediated by metalloproteinase shedding. Serum levels of sE-cad have also been observed in subjects with Sjögren's syndrome. Serum levels of sE-cad have also been observed in subjects with endometriosis. Serum levels of sE-cad have also been observed in subjects with systemic inflammatory response syndrome and multiorgan dysfunction syndrome. As disclosed herein, neurodegenerative disease and neural stress can also result in accumulation of the soluble ectodomain of E-cadherin in a bodily fluid.

### 1. Diagnosing Neurodegenerative Disease

Thus, provided herein is a method of diagnosing a neurodegenerative disease in a subject, comprising detecting the level of soluble E-cadherin (sE-cad) in a sample from the subject and comparing it to a reference standard.

In some aspects of the method, the subject has not been diagnosed with a cancer. In some aspects of the method, the subject has been diagnosed as not having a cancer.

In some aspects of the method, the subject has not been diagnosed with an autoimmune disease such as Sjögren's syndrome. In some aspects of the method, the subject has been diagnosed as not having an autoimmune disease such as Sjögren's syndrome.

In some aspects of the method, the subject has not been diagnosed with endometriosis. In some aspects of the method, the subject has been diagnosed as not having endometriosis.

In some aspects of the method, the subject has not been diagnosed with systemic inflammatory response syndrome or multiorgan dysfunction syndrome. In some aspects of the method, the subject has been diagnosed as not having systemic inflammatory response syndrome or multiorgan dysfunction syndrome.

Examples of neurodegenerative disorders include Alexander disease, Alper's disease, Alzheimer disease, Amyotrophic lateral sclerosis, Ataxia telangiectasia, Batten disease (also known as Spielmeyer-Vogt-Sjogren-Batten disease), Canavan disease , Cockayne syndrome, Corticobasal degeneration, Creutzfeldt-Jakob disease, Huntington disease, Kennedy's disease, Krabbe disease, Lewy body dementia, Machado-Joseph disease, Spinocerebellar ataxia type 3, Multiple sclerosis, Multiple System Atrophy, Parkinson's disease, Pelizaeus-Merzbacher Disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tay-Sachs, Transmissible spongiform encephalopathies (TSE), and Tabes dorsalis.

The condition or disease can in some aspects be Alzheimer's disease. Alzheimer's disease is a progressive neurodegenerative disorder that is characterized by the formation of senile plaques and neurofibrillary tangles containing amyloid β (Aβ) peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

Approximately twenty million people worldwide suffer with dementia that results from Alzheimer's disease. The disease can be early onset affecting individuals as young as 30 years of age, or it can be familial or sporadic. Familial Alzheimer's disease was once thought to be inherited strictly as an autosomal dominant trait; however, this view is changing as more genetic determinants are isolated. For example, some normal allelic variants of apolipoprotein E (ApoE), which is found in senile plaques, can either protect against or increase the risk of developing the disease.

Amyloid-β (Aβ) peptides are metabolites of the Alzheimer's disease-associated precursor protein, β-amyloid precursor protein (APP), and are believed to be the major pathological determinants of Alzheimer's disease (AD). These peptides consist mainly of 40 to 42 amino acids, Aβ1-40 (“Aβ40”) and Aβ1-42 (“Aβ42”), respectively. Aβ40 and Aβ42 are generated by two enzymatic cleavages occurring close to the C-terminus of APP. The enzymes responsible for the cleavage, β-secretase and γ-secretase, generate the—and C-termini of Aβ, respectively. The amino terminus of Aβ is formed by β-secretase cleavage between methionine residue 596 and aspartate residue 597 of APP (APP 695 isoform numbering).

In normal individuals, the Aβ peptide is found in two predominant forms, the majority Aβ-40 (also known as Aβ1-40) form and the minority Aβ42 (also known as Aβ1-42) form, each having a distinct COOH-terminus. The major histological lesions of AD are neuritic plaques and neurofibrillary tangles occurring in affected brain regions. Neuritic plaques consist of Aβ peptides, primarily Aβ40 and Aβ42. Although healthy neurons produce at least ten times more Aβ40 compared to Aβ42, plaques contain a larger proportion of the less soluble Aβ42. Patients with the most common form of familial Alzheimer's disease show an increase in the amount of the Aβ42 form. The Aβ40 form is not associated with early deposits of amyloid plaques. In contrast, the Aβ42 form accumulates early and predominantly in the parenchymal plaques and there is strong evidence that Aβ42 plays a major role in amyloid plaque deposits in familial Alzheimer's disease patients.

Symptoms of Aβ-related disorders are well known to those of skill in the art. For example, symptoms of Alzheimer's disease are well known in the art and can include, e.g., memory loss, mild cognitive impairment, cognitive decline, severe cognitive impairment and personality changes that result in loss of functional ability, e.g., over the course of a decade. In debilitated states, patients usually exhibit severe impairment, and retain only vegetative neurologic function. Symptoms of Alzheimer's disease can also include certain art-known neuropathological lesions, including intracellular neurofibrillary tangles and extracellular parenchymal and cerebrovascular amyloid.

### 2. Detecting Neuronal Stress

Also disclosed is a method of detecting neuronal stress in a subject, comprising detecting soluble E-cadherin (sE-cad) in a sample from the subject. As used herein “neuronal stress” or “neural stress” is used herein to refer to any damage, disease, or condition of the nervous system.

Neuronal stress can occur due to a wide range of conditions, illnesses, injuries, and as a result of iatrogenesis. Possible causes of widespread (diffuse) neuronal stress include prolonged hypoxia (shortage of oxygen), poisoning by teratogens (including alcohol), infection, and neurological illness. Chemotherapy can cause damage to the neural stem cells and oligodendrocyte cells that produce myelin. Common causes of focal or localized neuronal stress are physical trauma (traumatic brain injury), stroke, aneurysm, surgery, or neurological illness.

Thus, in some aspects, the neuronal stress is the result of trauma, stroke, ischemia, viral infection, or bacterial infection. For example, the neuronal stress can be the result of encephalitis or meningitis.

As disclosed herein, E-cadherin shedding resulting from neural stress can be transient. Thus, in some aspects sE-cad levels can normalize over time. For example, a transient change is sE-cad can occur following, for example, trauma, stroke, or infection. Thus, in some aspects, the method involves detecting sE-cad levels within 1, 2, 3, 4, 5, 6, 7, 8, 9, 10, 11, 12, 13, 14, 15, 16, 17, 18, 19, 20, 25, 30 days of a known or suspected neural event. In some aspects, the method involves taking two or more measurements over a time period to determine the rate of change in sE-cad levels.

### 3. Monitoring Severity

Also provided is a method of monitoring the severity of a neurodegenerative disease in a subject, comprising comparing levels of soluble E-cadherin (sE-cad) in a sample obtained from the subject at multiple time points, wherein detectable change in sE-cad levels in samples at later time points indicates a change in the severity of the neurodegenerative disease. Thus, in some aspects, a detectable increase in sE-cad levels in samples at later time points indicates the neurodegenerative disease is increasing in severity, wherein a decrease in sE-cad levels in samples at later time points indicates the neurodegenerative disease is decreasing in severity.

### 4. Monitoring Response to Treatment

Also provided is a method of monitoring a response to a neurodegenerative disease treatment in a subject, the method comprising comparing levels of soluble E-cadherin (sE-cad) in a sample obtained from the subject at multiple time points during treatment of the subject, wherein a detectable change in sE-cad levels in samples at later time points corresponds to efficacy of treatment. Thus, in some aspects, a detectable decrease in sE-cad levels in samples at later time points indicates effective treatment.

The treatment can be any known or recently discovered therapeutic composition or method designed or discovered to treat or ameliorate a neurodegenerative disease.

For example, the treatment can comprise a neuroprotective agent. For example, the neuroprotective agent can be an acetylcholinesterase inhibitor, a glutamatergic receptor antagonist, HDAC inhibitors, an anti-inflammatory agent, or divalproex sodium. Thus, the treatment can comprise a cholinesterase inhibitor. Non-limiting examples of cholinesterase inhibitors include galantamine, rivastigmine, donepezil and tacrine.

The treatment can comprise an N-methyl D-aspartate (NMDA) antagonist. For example, the treatment can comprise memantine.

The treatment can comprise a non-ATP Competitive Glycogen Synthase Kinase 3β (GSK-3β) Inhibitor. For example, the treatment can comprise thiadiazolidinones (TDZD).

The treatment can comprise an anti-inflammatory drug. Non-limiting examples of anti-inflammatory drugs include NSAIDS, COX inhibitors (e.g., celecoxib, etodolac), and EP receptor agonists/antagonists.

The treatment can comprise a statin (HMG-CoA reductase inhibitors). For example, the treatment can comprise Atorvastatin, Cerivastatin, Fluvastatin, Lovastatin, Mevastatin, Pitavastatin, Pravastatin, Rosuvastatin, Simvastatin, Simvastatin+Ezetimibe, Lovastatin+Niacin extended-release, or Atorvastatin+Amlodipine Besylate.

The treatment can comprise a drug affecting the cleavage of beta or gamma-secretase activity. For example, the treatment can comprise LY450139 (phase III clinical trials).

The treatment can comprise a selective Aβ42-lowering agent. For example, the treatment can comprise Tarenflurbil (Flurizan) (phase III trials).

The treatment can comprise a means of preventing amyloid accumulation. For example, the treatment can comprise an Aβ vaccine, Pittsburgh Compound-B, or intravenous immunoglobulin. Thus, the treatment can comprise bapineuzumab (Phase II clinical trials).

The treatment can comprise an anti-aggregation agent. For example, the treatment can comprise tramiprosate.

The treatment can comprise a dopamine agonist. For example, the dopamine agonist can be levodopa.

### 5. Measuring sE-cad

The herein disclosed method can comprise detecting sE-cad levels in a sample from the subject.

The sE-cad can be a fragment of an E-cadherin protein. In some aspects, the fragment comprises the extracellular domain of E-cadherin. In some aspects, the E-cadherin is mammalian. In some aspects, the E-cadherin is human E-cadherin. Thus, in some aspects, the sE-cad has the amino acid sequence SEQ ID NO:4. Thus, in some aspects, the sE-cad has the amino acid sequence SEQ ID NO:4.

In some aspects, the sE-cad fragment of E-cadherin can be detected by an antibody that specifically binds the extracellular domain of E-cadherin. Thus, in some aspects, the sE-cad can be detected by an antibody that specifically binds the amino acid sequence SEQ ID NO:4.

The sample can be a tissue sample or a bodily fluid. The bodily fluid can be, for example, blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. The tissue can be any tissue of the central or peripheral nervous system. Thus, the tissue can be obtained from, for example, the brain.

The method can further comprise comparing the levels of sE-cad to one or more reference standard.

In some aspects, the reference standard represents the level of sE-cad in a sample from one or more individuals that do not have a neurodegenerative disease or neuronal stress. Thus, in some aspects, a higher level of sE-cad in the sample from the subject than the reference standard indicates that the subject has the neurodegenerative disease or neuronal stress. In some aspects, a measurable decrease in the level of sE-cad in the sample from the subject over time indicates that the subject had a neuronal stress or is recovering from a neurodegenerative disease. In some aspects, a measurable increase in the level of sE-cad in the sample from the subject over time indicates that the subject has a neurodegenerative disease that is advancing in severity.

In some aspects, the reference standard represents the level of sE-cad in a sample from one or more individuals having a neurodegenerative disease or neuronal stress. Thus, in some aspects, a level of sE-cad in the sample from the subject equivalent to that of the reference standard indicates that the subject has the neurodegenerative disease or neuronal stress.

In some aspects, the method comprises comparing the level of sE-cad to at least a first and second reference standard, wherein the first reference standard represents the level of sE-cad in a sample from one or more individuals that do not have a neurodegenerative disease or neuronal stress, and where the second reference standard represents the level of sE-cad in a sample from one or more individuals having a neurodegenerative disease or neuronal stress.

In some aspects, reference standards have been used to establish reference concentration ranges whereby a user can compare the levels of sE-cad detected in a sample to these reference ranges.

In some aspects, the sE-cad of the disclosed method consists of a protein fragment of E-cadherin, wherein antibodies specific for the extracellular domain of E-cadherin can specifically bind the sE-cad.

i. Immunodetection

Soluble E-cadherin can be measured in the sample from the subject using routine methods of protein detection, including immunodetection methods. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (Di1C18(5)); DIDS; Dihydorhodamine 123 (DHR); Dil (Di1C18(3)); I Dinitrophenol; DiO (DiOC18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl)quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immuno analysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions.

Gel shift methods can include using, for example, colloidal forms of COOMASSIE (Imperial Chemicals Industries, Ltd) blue stain to detect proteins in gels such as polyacrylamide electrophoresis gels. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than lmm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### C. KITS

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting sE-cad, the kit comprising a solid surface, a means of capturing sE-cad from a sample onto the solid surface, and a means of detecting the captured sE-cad. The kits also can contain solutions for diluting the sample and/or washing the solid surface. The disclosed kits can also include instructions for conducting an assay with the kit.

### D. USES

The disclosed compositions can be used in a variety of ways as research tools. Other uses are disclosed, apparent from the disclosure, and/or will be understood by those in the art.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

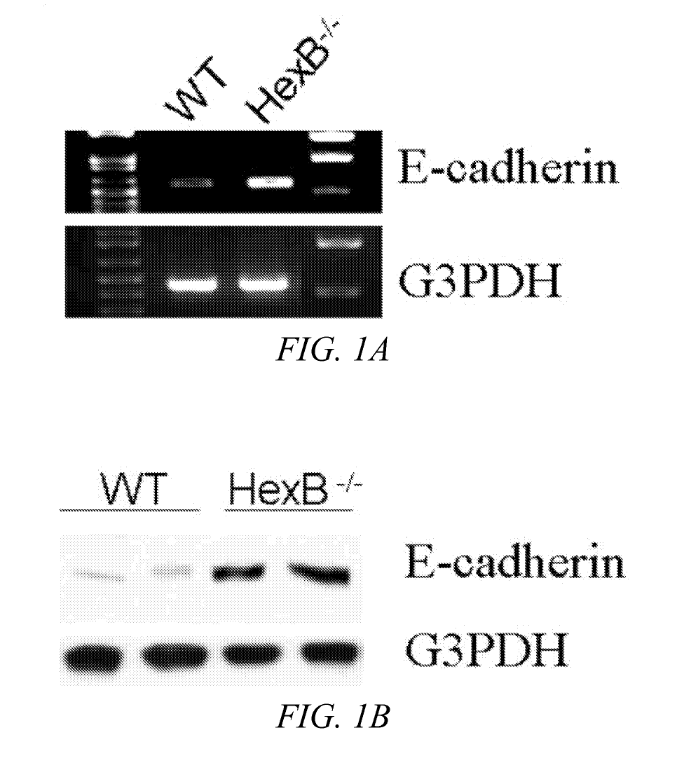

As shown in FIG. 1, E-cadherin transcript levels are dramatically increased in HexB^{-/-} mice. Semi-quantitative RT-PCR was used to examine levels of E-cadherin in 4 month old brains from wild-type (WT) and HexB^{-/-} mice (FIG. 1A). E-cadherin mRNA was greatly induced in 4 month old HexB^{-/-} mice compared to age-matched littermate WT controls. At 4 months of age, HexB^{-/-} mice clinically exhibit signs of neurodegeneration including muscle weakness, rigidity and motor deterioration. WT and HexB^{-/-} brains were resected at 4 months of age and processed for E-cadherin levels by Western immunoblotting (FIG. 1B).

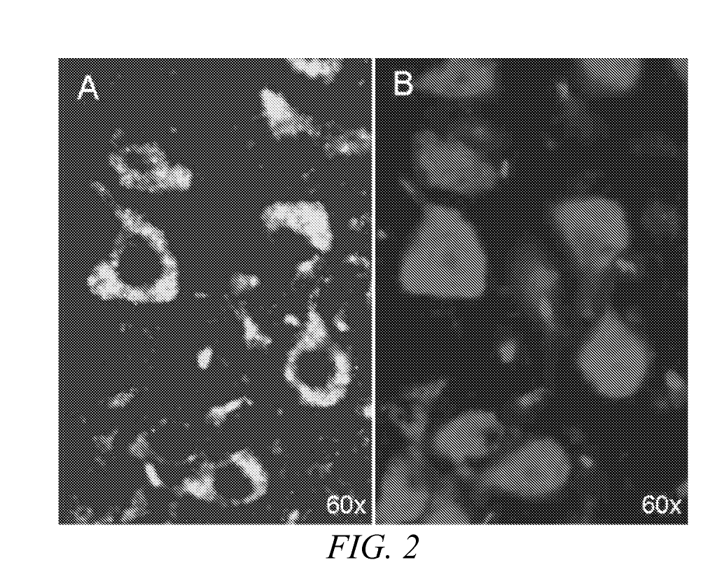

E-cadherin co-localized to neurons of HexB^{-/-} mice (FIG. 2). The identity of brain cells expressing E-cadherin in HexB^{-/-} mice was examined by double immunofluorescence. E-cadherin (FIG. 2A) was located in a perinuclear/cytoplasmic distribution of Neun-positive (FIG. 2B) neurons. Slides were examined on an Olympus fluorescent microscope.

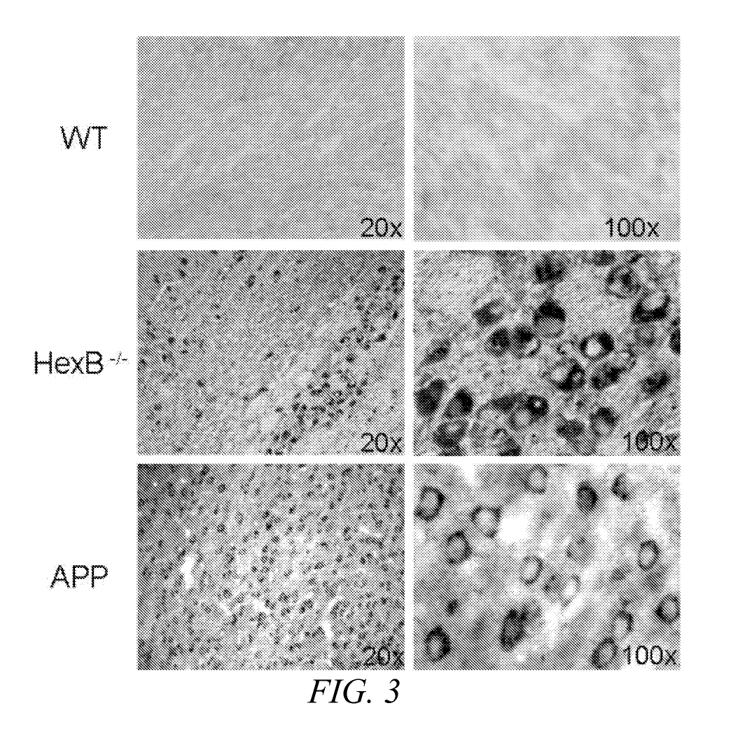

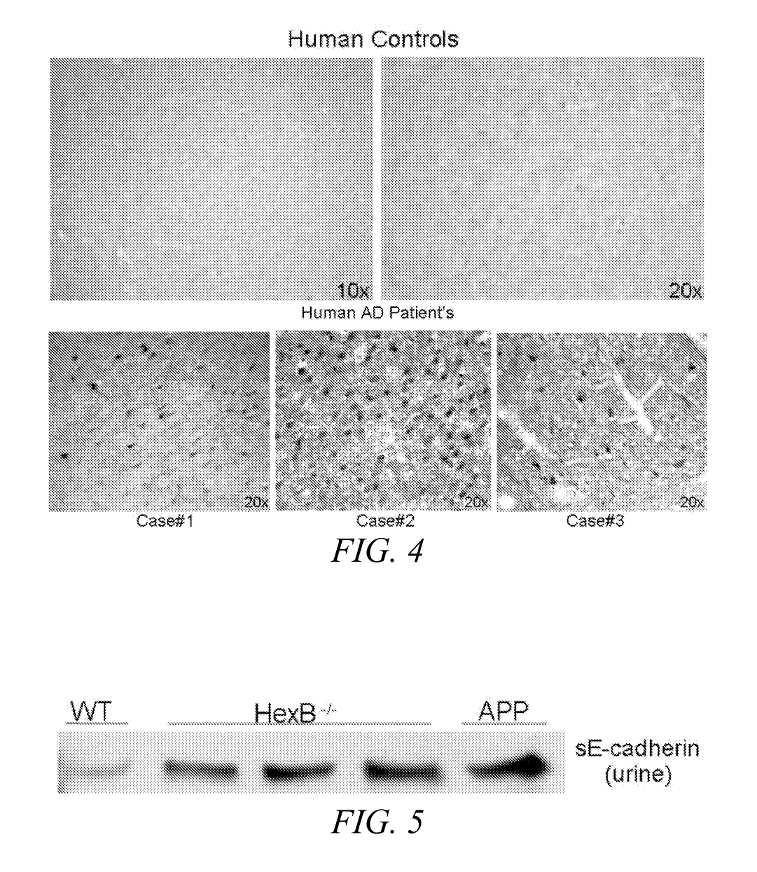

Brains of 4 month old age-matched WT and HexB^{-/-} mice and 8 month APP mice were immunostained for E-cadherin (FIG. 3; 20-100× magnification). Likewise, brains of human Alzheimers Disease Patient's and age-matched human controls were immunostained for E-cadherin (FIG. 4; 10-20× magnification).

E-cadherin ectodomain shedding was further observed in the urine of Tay Sachs and Alzheimers (APP) mice that exhibit signs of neurodegeneration (FIG. 5). Urine from 4 month old Tay sachs and 8 month old Alzheimers (APP) mice was collected and tested for E-cadherin fragments (sE-cad) using an antibody specific for the extracellular epitope of E-cadherin. An 80 kDa protein was detected in the urine of these mice, indicating proteolytical shedding of the protein.

### F. REFERENCES

Banks R E, Porter W H, Whelan P, Smith P H, Selby P J. Soluble forms of the adhesion molecule E-cadherin in urine. J Clin Pathol. 1995 February; 48(2):179-80.
Billion K, Ibrahim H, Mauch C, Niessen C M. Increased soluble E-cadherin in melanoma patients. Skin Pharmacol Physiol. 2006; 19(2):65-70. Epub 2006 May 9.
Charalabopoulos K, Gogali A, Dalavaga Y, Daskalopoulos G, Vassiliou M, Bablekos G, Karakosta A, Constantopoulos S. The clinical significance of soluble E-cadherin in nonsmall cell lung cancer. Exp Oncol. 2006 March; 28(1):83-5.
De Wever O, Derycke L, Hendrix A, De Meerleer G, Godeau F, Depypere H, Bracke M. Soluble cadherins as cancer biomarkers. Clin Exp Metastasis. 2007; 24(8):685-97.
Derycke L, De Wever O, Stove V, Vanhoecke B, Delanghe J, Depypere H, Bracke M. Soluble N-cadherin in human biological fluids. Int J Cancer. 2006 Dec. 15; 119(12):2895-900.
Fu C, Lang J. Serum soluble E-cadherin level in patients with endometriosis. Chin Med Sci J. 2002 June; 17(2):121-3.
Gil O D, Lee C, Ariztia E V, Wang F Q, Smith P J, Hope J M, Fishman D A. Lysophosphatidic acid (LPA) promotes E-cadherin ectodomain shedding and OVCA429 cell invasion in an uPA-dependent manner. Gynecol Oncol. 2008 February; 108(2):361-9
Johnson S K, Ramani V C, Hennings L, Haun R S. Kallikrein 7 enhances pancreatic cancer cell invasion by shedding E-cadherin. Cancer. 2007 May 1; 109(9):1811-20.
Jonsson M V, Salomonsson S, Øijordsbakken G, Skarstein K. Elevated serum levels of soluble E-cadherin in patients with primary Sjögren's syndrome. Scand J Immunol. 2005 December; 62(6):552-9.
Karayiannakis A J, Syrigos K N, Savva A, Polychronidis A, Karatzas G, Simopoulos C. Serum E-cadherin concentrations and their response during laparoscopic and open cholecystectomy. Surg Endosc. 2002 November; 16(11):1551-4.
Lee K H, Choi E Y, Hyun M S, Jang B I, Kim T N, Kim S W, Song S K, Kim J H, Kim J R. Association of extracellular cleavage of E-cadherin mediated by MMP-7 with HGF-induced in vitro invasion in human stomach cancer cells. Eur Surg Res. 2007; 39(4):208-15.
Matsumoto K, Shariat S F, Casella R, Wheeler T M, Slawin K M, Lerner S P. Preoperative plasma soluble E-cadherin predicts metastases to lymph nodes and prognosis in patients undergoing radical cystectomy. J Urol. 2003 December; 170(6 Pt 1):2248-52.
Nawrocki-Raby B, Gilles C, Polette M, Bruyneel E, Laronze J Y, Bonnet N, Foidart J M, Mareel M, Birembaut P. Upregulation of MMPs by soluble E-cadherin in human lung tumor cells. Int J Cancer. 2003 Jul. 20; 105(6):790-5.
Pittard A J, Banks R E, Galley H F, Webster N R. Soluble E-cadherin concentrations in patients with systemic inflammatory response syndrome and multiorgan dysfunction syndrome. Br J Anaesth. 1996 May; 76(5):629-31.
Protheroe A S, Banks R E, Mzimba M, Porter W H, Southgate J, Singh P N, Bosomworth M, Hamden P, Smith P H, Whelan P, Selby P J. Urinary concentrations of the soluble adhesion molecule E-cadherin and total protein in patients with bladder cancer. Br J Cancer. 1999 April; 80(1-2):273-8.
Shariat S F, Matsumoto K, Casella R, Jian W, Lerner S P. Urinary levels of soluble e-cadherin in the detection of transitional cell carcinoma of the urinary bladder. Eur Urol. 2005 July; 48(1):69-76.
Shirahama S, Furukawa F, Wakita H, Takigawa M. E- and P-cadherin expression in tumor tissues and soluble E-cadherin levels in sera of patients with skin cancer. J Dermatol Sci. 1996 October; 13(1):30-6.
Shtutman M, Levina E, Ohouo P, Baig M, Roninson I B. Cell adhesion molecule L1 disrupts E-cadherin-containing adherens junctions and increases scattering and motility of MCF7 breast carcinoma cells. Cancer Res. 2006 Dec. 1; 66(23):11370-80.
Sundfeldt K, Ivarsson K, Rask K, Haeger M, Hedin L, Brännström M. Higher levels of soluble E-cadherin in cyst fluid from malignant ovarian tumours than in benign cysts. Anticancer Res. 2001 January-February; 21(1A):65-70.
Syrigos K N, Harrington K J, Karayiannakis A J, Baibas N, Katirtzoglou N, Roussou P. Circulating soluble E-cadherin levels are of prognostic significance in patients with multiple myeloma. Anticancer Res. 2004 May-June; 24(3b):2027-31.
Syrigos K N, Salgami E, Karayiannakis A J, Katirtzoglou N, Sekara E, Roussou P. Prognostic significance of soluble adhesion molecules in Hodgkin's disease. Anticancer Res. 2004 March-April; 24(2C):1243-7.
Wilmanns C, Grossmann J, Steinhauer S, Manthey G, Weinhold B, Schmitt-Gräff A, von Specht B U. Soluble serum E-cadherin as a marker of tumour progression in colorectal cancer patients. Clin Exp Metastasis. 2004; 21(1):75-8.

### G. SEQUENCES

| 1. SEQ ID NO: 1-mEcad | |
|---|---|
| 1 | madgprckrr kqanprrnnv tnyntvvean sdsddedklh iveeesitda adceggmpdd |
| 61 | elpadqtvlp ggsdrgggak ncwqdnvkdn ecdsdaeneq nhdpnveefl qqqdtaviyp |
| 121 | eapeedqrqg tpeasshden gtpdafsqll tcpycdrgyk rftslkehik yrheknednf |
| 181 | scslcsytfa yrtqlerhmt shksgreqrh vtqsggnrkf kctecgkafk ykhhlkehlr |
| 241 | ihsgekpyec pnckkrfshs gsysshissk kcislmpvng rprsglktsq csspslstsp |
| 301 | gsptrpqirq kienkplqep lsvnqiktep vdyefkpivv asgincstpl qngvfssggq |
| 361 | lqatsspqgv vqavvlptvg lvspisinls diqnvlkvav dgnvirqvle tnqaslaske |
| 421 | qeavsaspiq qgghsvisai slplvdqdgt tkiiinysle qpsqlqvvpq nlkkeipapt |
| 481 | nsckseklpe dltvksetdk sfegarddst cllcedcpgd lnalpelkhy dpecpaqppp |
| 541 | papatekpes sassagngdl spsqpplknl lsllkayyal naqpsteels kiadsvnlpl |
| 601 | dgvkkwfekm qagqipgqsp dppspgtgsv niptktdeqp qpadgnepqe dstrgqspvk |
| 661 | irsspvlpvg samngsrsct sspsplnlcs arnpqgyscv aegaqeepqv epldlslpkq |
| 721 | qgellerstv ssvyqnsvys vqeeplnlsc akkepqkdsc vtdsepvvnv vppsanpini |
| 781 | aiptvtaqlp tivaiadqns vpclralaan kqtilipqva ytysatvspa vqeppvkviq |
| 841 | pngnqderqd tssegvstve dqndsdstpp kkktrkteng myacdlcdki fqksssllrh |
| 901 | kyehtgkrph ecgicrkafk hkhhliehmr lhsgekpyqc dkcgkrfshs gsysqhmnhr |
| 961 | ysyckrgaed rdameqedag pevlpevlat ehvgarasps qadsderesl treededsek |
| 1021 | eeeeedkeme elqegkecen pqgeeeeeee eeeeeeeeee eeveadeaeh eaaaktdgtv |
| 1081 | evgaaqqags leqkasesem eseseseqls eektnea |
| 2. SEQ ID NO: 2-mEcad extracellular domain | |
| 1 | madgprckrr kqanprrnnv tnyntvvean sdsddedklh iveeesitda adceggmpdd |
| 61 | elpadqtvlp ggsdrgggak ncwqdnvkdn ecdsdaeneq nhdpnveefl qqqdtaviyp |
| 121 | eapeedqrqg tpeasshden gtpdafsqll tcpycdrgyk rftslkehik yrheknednf |
| 181 | scslcsytfa yrtqlerhmt shksgreqrh vtqsggnrkf kctecgkafk ykhhlkehlr |
| 241 | ihsgekpyec pnckkrfshs gsysshissk kcislmpvng rprsglktsq csspslstsp |
| 301 | gsptrpqirq kienkplqep lsvnqiktep vdyefkpivv asgincstpl qngvfssggq |
| 361 | lqatsspqgv vqavvlptvg lvspisinls diqnvlkvav dgnvirqvle tnqaslaske |
| 421 | qeavsaspiq qgghsvisai slplvdqdgt tkiiinysle qpsqlqvvpq nlkkeipapt |
| 481 | nsckseklpe dltvksetdk sfegarddst cllcedcpgd lnalpelkhy dpecpaqppp |
| 541 | papatekpes sassagngdl spsqpplknl lsllkayyal naqpsteels kiadsvnlpl |
| 601 | dgvkkwfekm qagqipgqsp dppspgtgsv niptktdeqp qpadgnepqe dstrgqspvk |
| 661 | irsspvlpvg samngsrsct sspsplnlcs arnpqgyscv aegaqeep |
| 3. SEQ ID NO: 3-hEcad | |
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa svsgiqaell tfpnsspglr rqkrdwvipp iscpenekgp fpknlvqiks |
| 181 | nkdkegkvfy sitgqgadtp pvgvfiiere tgwlkvtepl dreriatytl fshavssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnptty kgqvpenean vvittlkvtd adapntpawe avytilnddg |
| 421 | gqfvvttnpv nndgilktak gldfeakqqy ilhvavtnvv pfevslttst atvtvdvldv |
| 481 | neapifvppe krvevsedfg vgqeitsyta qepdtfmeqk ityriwrdta nwleinpdtg |
| 541 | aistraeldr edfehvknst ytaliiatdn gspvatgtgt lllilsdvnd napipeprti |
| 601 | ffcernpkpq viniidadlp pntspftael thgasanwti qyndptqesi ilkpkmalev |
| 661 | gdykinlklm dnqnkdqvtt levsvcdceg aagvcrkaqp veaglqipai lgilggilal |
| 721 | lililllllf lrrravvkep llppeddtrd nvyyydeegg geedqdfdls qlhrgldarp |
| 781 | evtrndvapt lmsvprylpr panpdeignf idenlkaadt dptappydsl lvfdyegsgs |
| 841 | eaaslsslns sesdkdqdyd ylnewgnrfk kladmyggge dd |
| 4. SEQ ID NO: 4-hEcad extracellular domain | |
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa svsgiqaell tfpnsspglr rqkrdwvipp iscpenekgp fpknlvqiks |
| 181 | nkdkegkvfy sitgqgadtp pvgvfiiere tgwlkvtepl dreriatytl fshavssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnptty kgqvpenean vvittlkvtd adapntpawe avytilnddg |
| 421 | gqfvvttnpv nndgilktak gldfeakqqy ilhvavtnvv pfevslttst atvtvdvldv |
| 481 | neapifvppe krvevsedfg vgqeitsyta qepdtfmeqk ityriwrdta nwleinpdtg |
| 541 | aistraeldr edfehvknst ytaliiatdn gspvatgtgt lllilsdvnd napipeprti |
| 601 | ffcernpkpq viniidadlp pntspftael thgasanwti qyndptqesi ilkpkmalev |
| 661 | gdykinlklm dnqnkdqvtt levsvcdceg aagvcrkaqp veaglqip |

What is claimed is:

1. A method of diagnosing a neurodegenerative disease or neuronal stress in a subject, comprising detecting the level of soluble E-cadherin (sE-cad) in a sample from the subject and comparing it to one or more reference standards.

2. The method of claim 1, wherein the reference standard represents the level of sE-cad in a sample from one or more individuals that do not have the neurodegenerative disease, wherein a higher level of sE-cad in the sample from the subject than the reference standard indicates that the subject has the neurodegenerative disease.

3. The method of claim 1, wherein the reference standard represents the level of sE-cad in a sample from one or more individuals having the neurodegenerative disease, wherein a level of sE-cad in the sample from the subject equivalent to that of the reference standard indicates that the subject has the neurodegenerative disease.

4. The method of claim 1, wherein the neurodegenerative disease is Tay-Sachs.

5. The method of claim 1, wherein the neurodegenerative disease is Alzheimer's disease.

6. The method of claim 1, wherein the sE-cad is detected by an antibody that specifically binds the extracellular domain of E-cadherin.

7. The method of claim 5, wherein the antibody specifically binds the amino acid sequence SEQ ID NO:4.

8. The method of claim 1, wherein the neuronal stress is the result of trauma, stroke, ischemia, viral infection, or bacterial infection.

9. A method of monitoring the severity of a neurodegenerative disease in a subject, comprising comparing levels of soluble E-cadherin (sE-cad) in a sample obtained from the subject at multiple time points, wherein a change in sE-cad levels in samples at later time points indicates a change in severity of neurodegenerative disease.

10. The method of claim 9, wherein the neurodegenerative disease is Tay-Sachs.

11. The method of claim 9, wherein the neurodegenerative disease is Alzheimer's disease.

12. The method of claim 9, wherein the sE-cad is detected by an antibody that specifically binds the extracellular domain of E-cadherin.

13. The method of claim 11, wherein the antibody specifically binds the amino acid sequence SEQ ID NO:4.

14. A method of monitoring a response to a neurodegenerative disease treatment in a subject, the method comprising comparing levels of soluble E-cadherin (sE-cad) in a sample obtained from the subject at multiple time points during treatment of the subject, wherein a decrease in sE-cad levels in samples at later time points indicates effective treatment.

15. The method of claim 14, wherein the neurodegenerative disease is Tay-Sachs.

16. The method of claim 14, wherein the sE-cad is detected by an antibody that specifically binds the extracellular domain of E-cadherin.

17. The method of claim 16, wherein the antibody specifically binds the amino acid sequence SEQ ID NO:4.
